(19) United States
(12) Patent Application Publication
(10) Pub. No.: US 2007/0112291 A1
(43) Pub. Date: May 17, 2007

(54) Shunt system with coating and flow restricting component exerting a passive and essentially constant resistance to outflow

(75) Inventor: Svend Erik BØRGESEN, Kokkedal (DK)

Correspondence Address: BROWDY AND NEIMARK, P.L.L.C.;624 NINTH STREET, NW, SUITE 300, WASHINGTON, DC, 20001-5303, US

(73) Assignee: Sinu Shunt A/S, Glostrup (DK)

(21) Appl. No.: 10/580,650

(22) PCT Filed: Nov. 26, 2004
(86) PCT No.: PCT/DK04/00824

§ 371 Date: May 25, 2006

Related U.S. Application Data
(60) Provisional application No. 60/524,892, filed on Nov. 26, 2003.

(30) Foreign Application Priority Data
Nov. 26, 2003 (DK) PA 2003 01747

(51) Int. Cl. A61M 5/00

(52) U.S. Cl. 604/8

(57) ABSTRACT

The present invention relates to an improved cerebrospinal fluid shunt system comprising a coating covering at least part of the system and a flow restricting component exerting a passive and essentially constant resistance to flow. The present invention also relates to methods for implanting different catheters of a cerebrospinal fluid shunt system into a brain ventricle and the sinus system, respectively, of an individual. The present invention further relates to methods for shunting cerebrospinal fluid from a brain ventricle to the sinus system of an individual.

All patent and non-patent references cited in the present patent application is hereby incorporated in their entirety. This application is a non-provisional of U.S. provisional application Ser. No. 60/524,892 filed 26 Nov. 2003, which is hereby incorporated by reference in its entirety.

### FIELD OF INVENTION

The present invention relates to an improved cerebrospinal fluid shunt system comprising a coating covering at least part of the system and a flow restricting component exerting a passive and essentially constant resistance to flow. The coated shunt system is more resilient to wear and is better suited for implantation into the ventricles and the sinus system of the brain than conventional shunt systems. The passive and essentially constant resistance to outflow eliminates the need for using pressure sensitive valves and other mechanical components which are sensitive to wear.

The present invention also relates to methods for implanting different catheters of a cerebrospinal fluid shunt system into a brain ventricle and the sinus system, respectively, of an individual. The present invention further relates to methods for shunting cerebrospinal fluid from a brain ventricle to the sinus system of an individual.

### BACKGROUND OF INVENTION

Cerebrospinal Fluid

The brain and spinal cord are bathed in cerebrospinal fluid (CSF) and encased within the cranium and vertebral column inside a thin membrane known as the meninges. The space within the meninges includes the subarachnoid space, the ventricles (including the lateral ventricle, third ventricle, and fourth ventricle), the vertebral column, and the brain interstitial spaces. The volume of the brain intracranial spaces is on average about 1700 ml. The volume of the brain is approximately 1400 ml, and the volume of the intracranial blood is approximately 150 ml. The remaining 150 ml is filled with CSF (this volume may vary within 60 ml to 290 ml). The CSF circulates within the CSF space. Cerebrospinal fluid is formed in the ventricular system irrespective of the intracranial pressure (ICP). The formation rate is constant, with a range of 0.3-0.4 ml/min. (Bo/rgesen and Gjerris 1987).

Under normal conditions, the CSF is produced in the chorioid plexus in the ventricles. It flows through the ventricles, aqueduct and basal cisterns over the cerebral surface to the arachnoid villi, from where the CSF is absorbed into the sagittal sinus (including sinus transversus). The production and absorption of CSF are well described in the medical literature. See, e.g., Adams et al. (1989) “Principles of Neurology,” pp. 501-502.

Articles discussing pressures and other characteristics of CSF in the CSF space include Condon (1986) J. Comput. Assit. Tomogr. 10:784-792; Condon (1987) J. Comput. Assit. Tomogr. 11:203-207; Chapman (1990) Neurosurgery 26:181-189; Magneas (1976) J. Neurosurgery 44:698-705; Langfitt (1975) Neurosurgery 22:302-320.

Overview of Prior Art CSF Shunts

Prior art shunts have a number of disadvantages:
(a) Shunt infection—Most studies have reported shunt infection rates of the order of 5-10% (and significantly higher than this for neonatal shunts). The vast majority of shunt infections occur in the lumen. Most shunt infections occur in the first six months after the operation and the most common organisms are staphylococci (Staphylococcus epidermidis, 40%; Staphylococcus aureus, 20%). Other species seen include coryneforms, streptococci, enterococci, aerobic Gram-negative rods, and yeasts (Drake J M, Sainte-Rose C. The shunt book. New York: Blackwell Scientific, 1995). Unfortunately, once a shunt is infected, it is almost always necessary to remove it and insert a temporary external ventricular drain. Apart from the practical problems associated with the treatment of shunt infection, it has been shown that there is an increase in the development of loculated CSF compartments, impaired intellectual outcome, and death after shunt infection. (Drake J M, Sainte-Rose C. The shunt book. New York: Blackwell Scientific, 1995). Shunt infection may also be a contributory factor to seizures. One major factor causing infection is bacterial biofilm formation, which enhances antibiotic resistance and requires more than 1000 times higher levels of antibiotics than for non-biofilm bacterial infections.
(b) Mechanical failure—The use of Kaplan-Meier curves to display shunt survival has led to a far greater understanding of shunt failure, the symptoms of which are nonspecific and include fever, nausea, vomiting, irritability and malaise. Virtually all the studies to date have shown an exponential curve, with about 40% of shunts failing (including infection) in the first year and then about 5% a year. Over 50% of first shunt failures are due to obstruction, with the vast majority of these occurring at the ventricular catheter. This is almost certainly due to an overdrainage caused by many shunt systems, the consequence being that the ventricular catheter comes to lie against the ependyma and choroid plexus of the ventricle, and these tissues can then become incorporated into and block the holes at the end of the catheter. Other shunt malfunctions include fracturing of the tubing (the cause of about 15% of primary shunt malfunctions), migration of part or all of the shunt (7.5%) and problems with overdrainage (7%).
(c) Functional failure—The cause of functional failure is usually overdrainage. The underlying problem is one of siphoning from the ventricle to unphysiological resorption sites, usually the peritoneum. This overdrainage can result in subdural haematoma, low pressure symptoms (postural headache and nausea), and craniosynostosis. In an attempt to overcome the problem of siphoning, several attempts have been made to modify the performance of shunt valves. At present, shunt types can be broadly classified as follows:
   Differential-pressure valves (ball-in-spring, diaphragm, mitre or slit valves). The valves open at a pressure differential across the valve that is determined by the valve characteristics and is designated low, medium, or high (typically 5, 10, and 15 cm H₂O respectively). Some valves are programmable to allow the pressure setting to be altered after implantation
   Differential-pressure valves with an integral or inline antisiphon device
   Valves that regulate by flow rather than by pressure differentiation.

Furthermore, when conventional shunts drain to the abdomen (ventriculo-peritoneal shunts), fluid may accumulate in the abdomen and/or abdominal organs may be injured.
(d) Obstruction—Obstruction, a common problem, usually occurs when something clogs the ventricular catheter. Suboptimal placement can result in the catheter being clogged by brain cells or the choroid plexus. Tumor cells and protein buildups can also cause obstruction, as the cells adhere to the sides of the shunt. Obstruction can be complete, partial, or intermittent. Shunt obstruction will produce symptoms of increased. If the blockage is only partial or intermittent, the patient may experience periodic headaches, nausea and vomiting, drowsiness, listlessness, loss of appetite, and a general decrease in mental functioning. Complete obstruction can cause these same symptoms, including the more severe signs of blurred vision, loss of coordination, and possible loss of consciousness.
(e) Disconnection—shunt disconnection can occur at any point along the shunt, but is most common where the catheters connect to the shunt valve. The shunt may also break. Both these problems are due to mechanical weakness of the materials used.
(f) Shunt rejection—Another common problem with shunts is that ions or particles from the shunt may enter the body and cause shunt rejection or inflammation. Serious and long-term complications of shunt implantation may also include bleeding under the outermost covering of the brain (subdural hematoma). Metals and other less biocompatible materials may encourage coagulation and (possibly fatal) blood clotting in conventional shunts, due to “recognition” of the foreign material by the individual's immune system.

Several prior art solutions to these problems have been proposed. Antibiotic treatment alone has been found to be effective sometimes in the treatment of infection by Streptococcus and Haemophilus and studies have shown that antibiotics are effective in prophylactic treatments (Langley J M et al. Efficacy of antimicrobial prophylaxis in placement of cerebrospinal fluid shunts: meta-analysis. Clin Infect Dis 1993; 17:98-103). However, infected shunts still may have to be surgically removed, which is clearly undesirable.

Tissue engineered shunts have been proposed to improve biocompatibility, however there are as yet unresolved problems with the polymer types, cell type, and cell densities used (Lee I-W et al. The living shunt: a tissue engineering approach in the treatment of hydrocephalus. Neurol Res 2000; 22:105-110)

Shunts have also been tested that are impregnated with antimicrobials to prevent bacterial catheter-related infection (Duration of activity of cerebrospinal fluid shunt catheters impregnated with antimicrobials to prevent bacterial catheter-related infection. Bayston R., Lambert E. J Neurosurg 87 247-251 1997), however one problem with this shunt design is that the antimicrobial effect is not necessarily as long-lasting as the implanted shunt.

Currently preferred materials for shunt lumen walls are chosen for their reasonable biocompatibility, such as silicone rubbers and other natural and synthetic rubber materials. However, these types of materials have recently been shown to have a higher likelihood of purulent infection (“Pathogenesis and Prevention of Catheter-Related Infection”, Sherertz, R J, speaker at the “Shunt Technology: Challenges and Emerging Directions” conference, National Naval Medical Center, Bethesda, Md., USA, Jan. 8, 1999), probably as neutrophils are caused to migrate differently, which also leads to a higher inflammatory index and increased complement activation within the patient. The current solution to this problem is to use antibiotics.

There is thus a need for novel shunt systems avoiding the above problems associated with previous shunts.

### SUMMARY OF THE INVENTION

The present invention relates to an improved cerebrospinal fluid shunt system comprising a coating covering at least part of the shunt system and a flow restricting component exerting a passive and essentially constant resistance to flow.

In addition to improved biocompatible properties, the passive and essentially constant resistance to outflow of the shunt systems according to the invention eliminates the need for using pressure sensitive valves and other mechanical components which are sensitive to wear over time.

As the shunt system according to the invention operates without any mechanical pressure sensitive valves, the shunt system is able to shunt CSF from the CSF space at considerable lower intracranial pressures than conventional ventriculo-peritoneal shunts operating with pressure sensitive valves being activated at a certain predetermined opening pressure.

Not only does the coated shunt system according to the invention have an improved biocompatibility, it is also more resilient to wear compared to conventional shunts, and the shunt system according to the invention is therefore better suited for implantation into the ventricles and the sinus system of the brain than conventional shunt systems. Also, the coating provides a more persistent boundary between the shunt system and the surrounding biological materials, thus avoiding leakage of shunt micro-particles to the surrounding biological material.

The preferred coatings disclosed herein have been developed with particular focus on implantation of the shunt system into the brain of an individual. The biological environment of the brain is significantly different from many other environments of the body. Hence, biocompatible materials developed for increasing the biocompatibility of implants in other parts of the body of an individual cannot indiscriminately be transferred to the highly specialized environment of the brain.

Also, biocompatibility in connection with the present invention shall also be understood in the context of the shunt system being able to drain CSF comprising a variety of toxic substances relating to a number of different clinical conditions as disclosed herein below in more detail. The biocompatible properties of the shunt system of the invention must therefore also take account for the presence of toxic substances in the CSF.

Apart from treating hydrocephalus, the shunt system of the present invention can also be used for treating clinical conditions such as e.g. Alzheimer's disease. Treatment of Alzheimer's disease is an example of a clinical condition capable of being treated by using the shunts of the invention to drain CSF comprising e.g amyloid plaque proteins from the CSF space of an individual.

In addition to Alzheimer's disease, the shunt systems according to the present invention will also be useful in treating other conditions resulting from the accumulation of toxic substances and resulting lesions in the patient's brain, such as e.g. Down's Syndrome, hereditary cerebral hemorrhage with amyloidosis of the Dutch-Type (HCHWA-D), epilepsy, narcolepsy, Parkinson's disease, polyneuropathies, multiple sclerosis, amyotrophic lateral sclerosis (ALS), myasthenia gravis, muscular dystrophy, dystrophy myotonic, other myotonic syndromes, polymyositis, dermatomyositis, brain tumors, Guillain-Barre-Syndrome, and the like.

The treatment of the above clinical diseases set new and currently unmet demands for the development of more resilient CSF shunt systems with improved biocompatible and hemocompatible properties. The shunt of the present invention meets these demands and further has simple design principles avoiding e.g. mechanical pressure sensitive valves which are sensitive to wear over time.

Down's Syndrome

In one preferred embodiment of the present invention, a shunt system for use in a method for treatment of Down's syndrome is provided. Nearly all patients with Down's syndrome develop Alzheimer's if they live into their 40s. This is probably due to the finding that APP is located on chromosome 21, a key chromosome in the genetic aberrations causing Down's syndrome patients. Thus, it is probable that Down's syndrome patients with genetic aberrations such as trisomy 21 will overproduce APP and have high levels of potentially toxic amyloid precursors in their CSF.

Hereditary Cerebral Hemorrhage with Amyloidosis of the Dutch-Type (HCHWA-D)

Hereditary cerebral haemorrhage with amyloidosis-Dutch type (HCHWA-D) is an autosomal dominant disorder, caused by a single base mutation in the APP gene, resulting in recurrent haemorrhagic strokes and dementia (Brain. 1997 December; 120 (Pt 12):2243-9. It is envisaged that HCHWA-D and similar diseases caused by mutations in the APP gene may be treated using the methods described herein.

Epilepsy

In another, equally preferred embodiment of the present invention, a shunt system for use in a method for treatment of epilepsy is provided. Epilepsy is the tendency to have repeated seizures that originate in the brain. There are various toxic factors that may act to increase the risk of seizure. Increased levels of messenger RNAs for neurotrophic factors have been detected in brains during kindling epileptogenesis (Ernfors P, et al., Neuron. 1991 July; 7(1):165-76) and this is hypothesised to contribute to the development of epileptic syndromes. Furthermore, increases in the levels of the excitory neurotransmitter glutamate, which in turn triggers increases in calcium ions to toxic levels, may also contribute to seizure occurrence.

Parkinson's Disease

In another, equally preferred embodiment of the present invention, a shunt system for use in a method for treatment of Parkinson's disease is provided. Two different alpha-synuclein mutations have been shown to be associated with autosomal-dominant Parkinson's disease (PD), and the discovery that alpha-synuclein is a major component of Lewy bodies and Lewy neurites, the pathological hallmarks of PD, confirmed its role in PD pathogenesis. Pathological aggregation of the protein might be responsible for neurodegeneration and soluble oligomers of alpha-synuclein are hypothesised to be even more toxic (Lucking C B and Brice, A, Alpha-synuclein and Parkinson's disease Cell Mol Life Sci. 2000 December; 57(13-14):1894-908).

Polyneuropathies

In another, equally preferred embodiment of the present invention, a shunt system for use in a method for treatment of polyneuropathies is provided. Polyneuropathies are defined herein as diseases of the nerves, which often take the form of a noninflammatory degenerative disease of nerves, usually caused by toxins. As an example of these toxic substance, in acute motor axonal neuropathy (AMAN) (Kornberg, A. J. and Pestronk, A., Muscle Nerve 17:100-104 (1994)) and Miller-Fisher syndrome (Chiba, A. et al., Ann. Neurol. 31:677-679 (1992)), antibodies directed against neural antigens, such as glycolipids, have been reported in 30% to 90% of patients. Methods disclosed of the present invention are envisaged as being capable of treating any polyneuropathy caused, or associated with, toxic substances.

Multiple Sclerosis

In another, equally preferred embodiment of the present invention, a shunt system for use in a method for treatment of Multiple Sclerosis is provided. The “pathogen-mediated” theory of multiple sclerosis postulates that pathogens are involved in the etiology of the disease, which has been supported by results showing an association between C. Pneumoniae in the CSF and Multiple Sclerosis (BioDrugs 2001; 15(3):199-206). Other diseases may also be linked to toxic substances, including myasthenia gravis, muscular dystrophy, polymyositis, dermatomyositis, dystrophy myotonic and other myotonic syndromes, Amyotrophic lateral sclerosis (ALS), brain tumors, Guillain-Barre-Syndrome, and the like.

Biocompatible Materials

It will be understood that a “biocompatible” material as defined herein is a material which, when inserted into the brain of an individual, is capable of being reasonably well tolerated by the individual's body, i.e said material does not trigger major immune reactions or acute phase responses.

Biocompatibility shall refer equally to materials characterized by an inert surface, such as diamond-like-carbon, preventing biological material from maintaining a longer lasting contact with the inert surface, as well as to a surface, such as a polymer, coated with a plurality of charged species, such as e.g. hydrophilic polyethylene glycols, capable of increasing in particular the hemocompatibility of the polymer. Longer lasting contact as used herein is a contact which results in undesirable attachment to the surface, normally longer lasting contact will be a contact lasting at least hours, such as at least weeks, for example months.

Preferred examples of biocompatible materials are disclosed herein below. Carbon comprising inert materials represent one preferred class of biocompatible materials.

Carbon forms a strongly bonded 3 dimensional network when deposited as a coating under energetic conditions. This amorphous coating has properties approaching those of diamond as regards hardness, friction, chemical inertness and atomic density hence the term diamond like carbon (DLC). DLC coatings can be produced by plasma assisted chemical vapour deposition from hydrocarbon precursor gases, the coatings contain carbon and hydrogen (to about 30%) and therefore consist of elements which are main constituents in living organisms. In vitro tests have shown DLC to be biocompatible (L A Thomson, F G Law, N Rushton, J Franks. Biomaterials 12, 37 (1991)) and in vivo tests indicate that the coating also has hemocompatible properties.

Because of its atomic density, the coating acts as an effective diffusion barrier preventing ions from the shunt entering the body and protecting the shunt from attack by the biological environment. Turbostratic carbons, like pyrolytic carbon, are a form of graphite that is stronger and more wear resistant. Turbostatic carbons such as “On-X Carbon” (made by the “Medical Carbon Research Institute”, MCRI) are highly hemocompatible.

Sputtered carbon coatings such as Graphit-iC give exceptional friction and wear results in simple laboratory tests against metal counterfaces, demonstrating a high load bearing capacity and operating well in water-based environments, as well as being biocompatible.

Many ceramics, such as titanium nitride (TiN), are also known to have beneficial biocompatible and non-stick properties. TiN has been shown in some in vitro tests to be even more hemocompatible than pyrolytic carbon.

Phosphatidyl choline di-ester is another highly biocompatible coating.

Teflon and the like are other non-stick biocompatible materials exhibiting non-stick properties.

The improved coated shunt system disclosed herein has been found to advantageously and surprisingly reduce many of the problems associated with current shunts. Metals and other less biocompatible materials encourage coagulation and (possibly fatal) blood clotting in conventional shunts, however biocompatible coatings allow the problem of “recognition” of the non-biocompatible material by the individual's body to be avoided.

It is envisaged that shunt infection may be reduced by the advantageous coatings on the shunt disclosed herein, which will reduce adhesion of cells and other biological matter to the shunt and improve biocompatibility with the patient. Mechanical failure is reduced by the simplistic shunt design, as there is a shorter distance in this system between the ventricles and resorption site, and also no need for complex pressure valves. Mechanical failure is also reduced by some coatings which themselves have advantageous structural properties. Mechanical failure is also reduced by the fact that the coating can be used to coat less biocompatible, but structurally stronger, materials. This may also lead to decreased disconnection problems. This innovation is also thought to reduce infection rates because there is a smaller length for the CSF to travel, which, for example, leads to a reduced surface for biofilm formation. The biocompatibility of the shunt coatings disclosed herein also lead to a reduction in shunt rejection in patients. Obstruction of the shunt is also reduced due to decreased adhesion of one or more of brain cells, the choroid plexus, tumor cells and protein buildups.

Functional failure is reduced by the use of the sagittal sinus or transverse sinus as a resorption site, which allows the pressure difference over the CSF shunt system to remain essentially constant. Thus, in contrast to many of the shunt types in use today, the present invention does not rely on control of flow via “pressure control”—instead it functions on an entirely different principle: maintenance of a constant resistance to CSF flow. This would not be the case for resorption sites such as the peritoneum. Furthermore, the pressure difference generated across the shunt is similar to the physiological pressure differences between the ventricles and the normal CSF resorption site, thus regulating the CSF flow to be within the normal range and avoiding hyperdrainage. Posture-related pressure changes across the shunt are also beneficially avoided.

From measurements in 333 patients (Bo/rgesen and Gjerris 1987) and 52 normal humans (Albeck, Bo/rgesen et al.) it has been possible to establish the relationship between CSF production rate (FR), intracranial pressure (ICP), pressure in the sagittal sinus (P_{SS}) and the resistance to outflow of CSF (Rₒᵤₜ):
ICP=FR×Rₒᵤₜ+P_{SS}

The relation between the intracranial pressure and the formation rate is linear, and the production rate measured was found to be 0.3 ml/min. (Bo/rgesen and Gjerris 1989). The detailed knowledge on CSF-dynamics, obtained in the laboratories at the Department of Neurosurgery, Rigshospitalet, Copenhagen, Denmark, has provided the necessary data which make it possible to define a CSF shunt system that imitates the normal, physiological drainage of CSF.

The present invention thus provides a shunt capable of diverting the CSF into its normal resorption site, and the pressure difference over the CSF shunt system used is similar to the physiological pressure differences between the ventricles and the resorption site, thus regulating the CSF flow to be within the normal range and avoiding complications like functional failure due to hyperdrainage.

An important feature of the method according to the present invention is the maintainence of an essentially constant resistance to flow within the shunt, said constant resistance to flow being independent of the orientation of said shunt main body means. This means that the resistance is independent of whether the person using the shunt system is standing up or lying down.

By using a shunt which exerts a substantially constant resistance to outflow at the normal level, and by using the sagittal and/or transverse sinus as the resorption site, the drainage of CSF is regulated by the normal pressure differences between the production and the resorption sites. Excessive increases of the intracranial pressure are paralleled by increases also in the sinus used as the resoprtion site, and the CSF outflow through the shunt is impeded by a resistance in the low to normal range. Overdrainage, which is the most frequent reason for shunt failure in conventional shunts, is thus also avoided.

By using the sagittal sinus or transverse sinus as the recipient site, physiological increases of the intracranial pressure will not increase the differential pressure over the shunt. Posture related changes in the differential pressure as seen in shunts leading the CSF to the right atrium of the heart or to the peritoneal cavity are completely avoided.

### DESCRIPTION OF DRAWINGS

FIG. 1 is a longitudinal sectional view of an embodiment of the shunt system used according to the invention,

FIG. 2 is a sectional view of the shunt body shown in FIG. 1,

FIG. 3 is an end view of the shunt body shown in FIG. 2,

FIG. 4 is a longitudinal sectional view of the shunt body taken at right angles to the section shown in FIG. 2,

FIG. 5 is a perspective view of the shunt body shown in FIGS. 2-4,

FIG. 6 is a partial cross-sectional view of the head of a person, in which the shunt system illustrated in FIGS. 1-5 has been installed,

FIG. 7 is a longitudinal sectional view of the head of a person, in which the shunt system illustrated in FIGS. 1-5 has been installed, and

FIG. 8 is a sectional view as that shown in FIG. 7, where the sinus catheter has been inserted in the transverse sinus.

FIG. 9 is a longitudinal sectional view of the head of a person, in which the shunt system illustrated in FIGS. 1-5 has been installed.

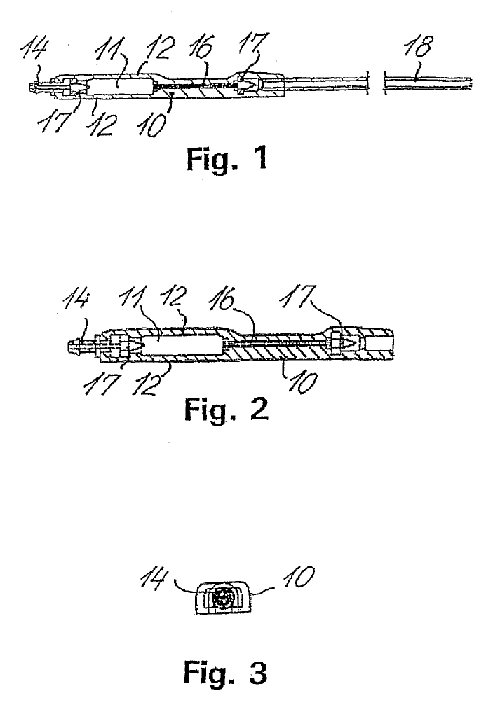

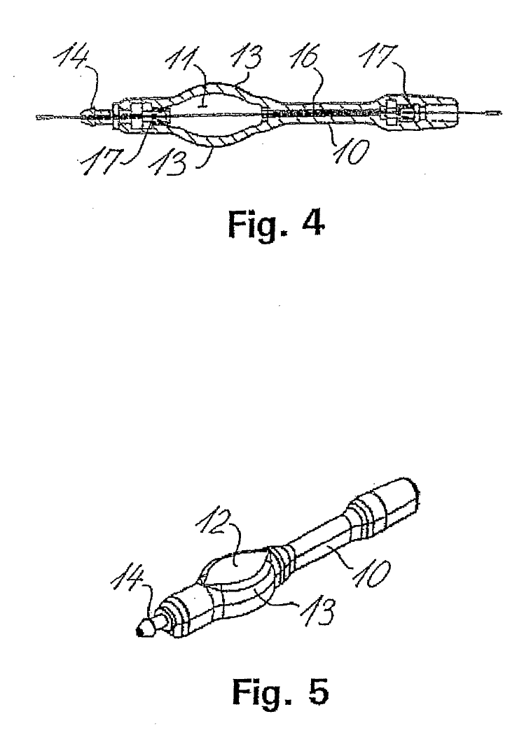

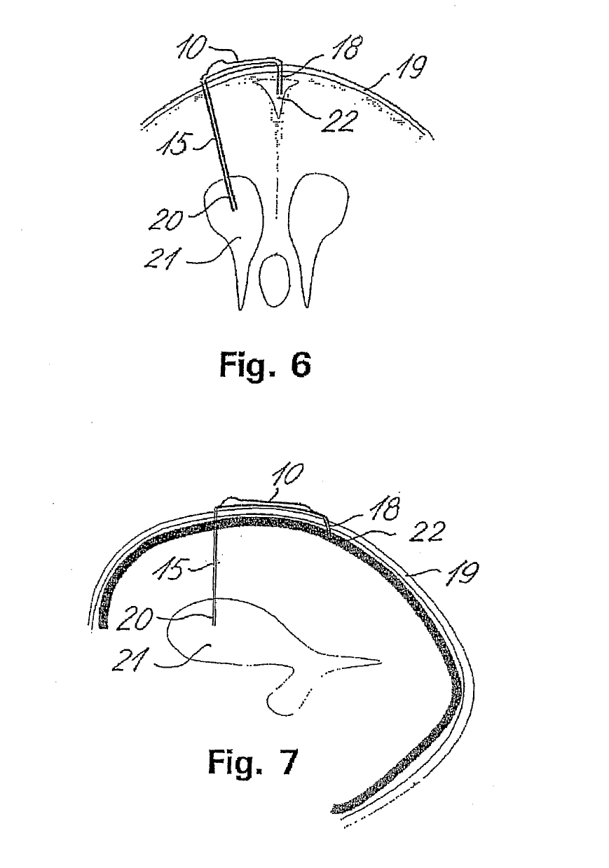

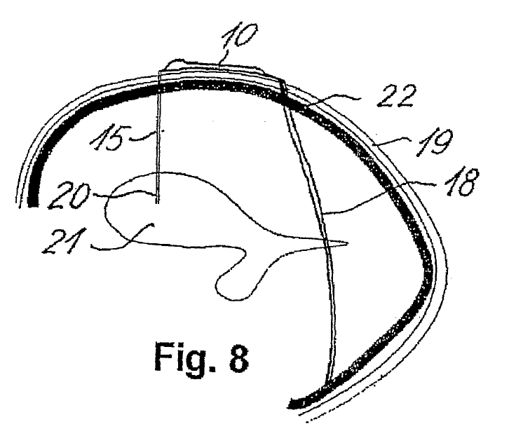

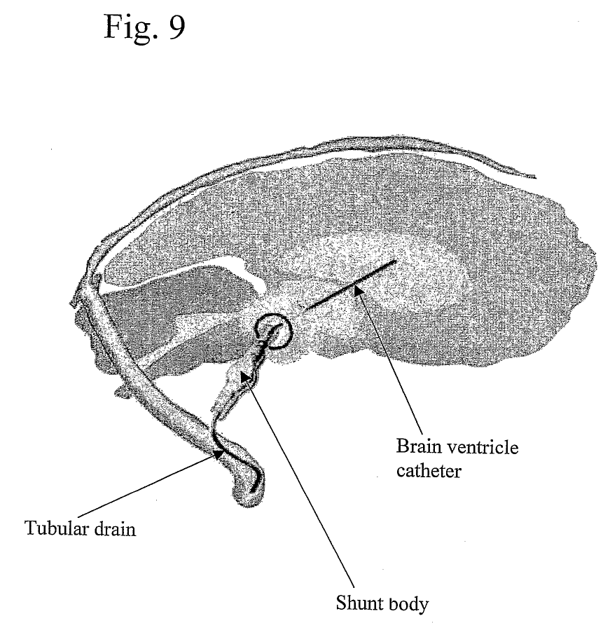

### DETAILED DESCRIPTION OF THE INVENTION

The shunt system provided in the present invention comprises a shunt body allowing fluid communication between a brain ventricle and a part of the sinus system of the individual. Said shunt body comprises a flow restricting component capable of maintaining a passive and essentially constant resistance to flow of cerebrospinal fluids through the shunt body. Preferably, said essentially constant resistance to flow of cerebrospinal fluids through the flow restricting component is of a constant value of less than 8 mm Hg/ml/min.

Said shunt system also comprises a brain ventricle catheter capable of being connected to the shunt body at a first location thereof. The brain ventricle catheter is capable of draining cerebrospinal fluids from a brain ventricle to the shunt body.

Said shunt system also comprises a sinus catheter capable of being connected to the shunt body at a second location thereof. Said sinus catheter is capable of draining to the sinus system of the individual cerebrospinal fluids having been drained from a brain ventricle and passed through the flow restricting component of the shunt body to the sinus catheter.

Either all or part of i) the internal or external surface of the shunt body, or ii) all or part of the internal or external surface of the brain ventricle catheter, or iii) all or part of the internal or external surface of the sinus catheter, can comprise a biocompatible and/or hemocompatible material comprising an inert surface preventing biological material from maintaining longer lasting contact with the inert surface, and/or comprising a hemocompatible surface coated with a plurality of charged species capable of increasing the hemocompatibility of the surface.

Accordingly, the internal or external surface of the shunt body, or the internal or external surface of the brain ventricle catheter, or the internal or external surface of the sinus catheter, can comprise a biocompatible and/or hemocompatible material comprising an inert surface preventing biological material from maintaining longer lasting contact with the inert surface, and/or comprise a polymer material coated with a plurality of charged species capable of increasing the hemocompatibility of the surface.

In one embodiment, the internal or external surface of the shunt body comprises a biocompatible and/or hemocompatible material comprising an inert surface preventing biological material from maintaining longer lasting contact with the inert surface, wherein the hemocompatible material can comprise a polymer material coated with a plurality of charged species capable of increasing the hemocompatibility of the surface.

In a further embodiment, the internal or external surface of the brain ventricle catheter also comprises a biocompatible and/or hemocompatible material comprising an inert surface preventing biological material from maintaining longer lasting contact with the inert surface, wherein the hemocompatible material can comprise a polymer material coated with a plurality of charged species capable of increasing the hemocompatibility of the surface.

In a still further embodiment, the internal or external surface of the sinus catheter also comprises a biocompatible and/or hemocompatible material comprising an inert surface preventing biological material from maintaining longer lasting contact with the inert surface, wherein the hemocompatible material can comprise a polymer material coated with a plurality of charged species capable of increasing the hemocompatibility of the surface.

The hemocompatible surface coated with a plurality of charged species capable of increasing the hemocompatibility of the surface can be e.g. a silicone elastomer, teflon, HD polyethylene, such as gas sterilized polypropylene, polysulfone, polystyrene, PVC, nylon, titanium, shape memory alloys such as Nitinol or polyethersulfone. The charged species can be e.g. polyethylene glycols or another macromolecule having a molecular weight of less than e.g. 20,000. The hemocompatible surface is in one embodiment a modified polymer surface as disclosed in PCT/DK00/00065 and/or PCT/DK01/00557.

The internal or external surfaces of the shunt system are preferably sterilisable. It is preferred that one or more of said surfaces act as an effective diffusion barrier preventing ions from the shunt entering the body and protecting the shunt from attack by the biological environment.

In another preferred embodiment of the present invention, one or more of said surfaces are non-adhesive. In another preferred embodiment, one or more of said surfaces are non-toxic. In another preferred embodiment, one or more of said surfaces are non-immunogenic.

In one preferred embodiment of the present invention, said biocompatible and/or hemocompatible material comprises diamond like carbon (DLC) or the like. Equally preferably, said biocompatible and/or hemocompatible material can comprise a turbostratic carbon, more preferably pyrolytic carbon.

In another preferred embodiment of the present invention, said biocompatible and/or hemocompatible material comprises a ceramic. Said ceramic is preferably titanium nitride (TiN), or the like. In another preferred embodiment, said biocompatible and/or hemocompatible material comprises phosphatidyl choline di-ester. In another preferred embodiment, said biocompatible and/or hemocompatible material comprises a Sputtered carbon coating, such as Graphit-iC or the like. In another preferred embodiment, said biocompatible and/or hemocompatible material comprises Teflon, and the like. In another embodiment of the present invention, said biocompatible and/or hemocompatible material comprises a calcification-resistant biocompatible material.

In one preferred embodiment, the surface is the external surface of the sinus catheter. In another preferred embodiment, the surface is the internal surface of the sinus catheter.

To carry out the method provided in the present invention, the brain ventricle catheter of the shunt is inserted a brain ventricle of an individual. Furthermore, the sinus catheter of the shunt system is inserted into the sinus system of said individual. Preferably, the brain ventricle catheter is connected to the shunt body at a first location thereof, and the sinus catheter is connected to the shunt body at a second location thereof, so that the shunt member provides fluidic communication between the first and second catheters. The final step in the method of the present invention comprises shunting cerebrospinal fluid present in a brain ventricle to the sinus system of the individual.

Flow Restricting Component

In one embodiment of the present invention, the flow restricting component is any structure capable of maintaining a passive and essentially constant resistance to CSF flow. Preferably, the flow restricting component of the shunt body is capable of maintaining a passive and essentially constant resistance to flow of cerebrospinal fluids through the shunt body of from 0.1 to less than 8 mm Hg/ml/min. In another preferred embodiment, the flow restricting component of the shunt body is capable of maintaining a passive and essentially constant resistance to flow of cerebrospinal fluids through the shunt body of from 0.5 to less than 8 mm Hg/ml/min. In another, equally preferred embodiment, the flow restricting component of the shunt body is capable of maintaining a passive and essentially constant resistance to flow of cerebrospinal fluids through the shunt body of from 1 to less than 8 mm Hg/ml/min. In another, equally preferred embodiment, the flow restricting component of the shunt body is capable of maintaining a passive and essentially constant resistance to flow of cerebrospinal fluids through the shunt body of from 2 to less than 8 mm Hg/ml/min. In another, equally preferred embodiment, the flow restricting component of the shunt body is capable of maintaining a passive and essentially constant resistance to flow of cerebrospinal fluids through the shunt body of from 3 to less than 8 mm Hg/ml/min. In another, equally preferred embodiment, the flow restricting component of the shunt body is capable of maintaining a passive and essentially constant resistance to flow of cerebrospinal fluids through the shunt body of from 4 to less than 8 mm Hg/ml/min. In another, equally preferred embodiment, the flow restricting component of the shunt body is capable of maintaining a passive and essentially constant resistance to flow of cerebrospinal fluids through the shunt body of from 6 to less than 8 mm Hg/ml/min. In another, equally preferred embodiment, the flow restricting component of the shunt body is capable of maintaining a passive and essentially constant resistance to flow of cerebrospinal fluids through the shunt body of from 0.1 to 7 mm Hg/ml/min. In another, equally preferred embodiment, the flow restricting component of the shunt body is capable of maintaining a passive and essentially constant resistance to flow of cerebrospinal fluids through the shunt body of from 0.1 to 6 mm Hg/ml/min. In another, equally preferred embodiment, the flow restricting component of the shunt body is capable of maintaining a passive and essentially constant resistance to flow of cerebrospinal fluids through the shunt body of from 0.1 to 5 mm Hg/ml/min. In another, equally preferred embodiment, the flow restricting component of the shunt body is capable of maintaining a passive and essentially constant resistance to flow of cerebrospinal fluids through the shunt body of from 0.1 to 4 mm Hg/ml/min. In another, equally preferred embodiment, the flow restricting component of the shunt body is capable of maintaining a passive and essentially constant resistance to flow of cerebrospinal fluids through the shunt body of from 0.1 to 3 mm Hg/ml/min. In another, equally preferred embodiment, the flow restricting component of the shunt body is capable of maintaining a passive and essentially constant resistance to flow of cerebrospinal fluids through the shunt body of from 0.1 to 2 mm Hg/ml/min. In another, equally preferred embodiment, the flow restricting component of the shunt body is capable of maintaining a passive and essentially constant resistance to flow of cerebrospinal fluids through the shunt body of from 0.1 to 1 mm Hg/ml/min. In another, equally preferred embodiment, the flow restricting component of the shunt body if capable of maintaining a passive and essentially constant resistance to flow of cerebrospinal fluids through the shunt body of from such as from 1 to 7 mm Hg/ml/min. In another, equally preferred embodiment, the flow restricting component of the shunt body is capable of maintaining a passive and essentially constant resistance to flow of cerebrospinal fluids through the shunt body of from 1 to 5 mm Hg/ml/min. In another, equally preferred embodiment, the flow restricting component of the shunt body is capable of maintaining a passive and essentially constant resistance to flow of cerebrospinal fluids through the shunt body of from 1 to 3 mm Hg/ml/min. In another, equally preferred embodiment, the flow restricting component of the shunt body is capable of maintaining a passive and essentially constant resistance to flow of cerebrospinal fluids through the shunt body of from 1 to 2 mm Hg/ml/min. In another, equally preferred embodiment, the flow restricting component of the shunt body is capable of maintaining a passive and essentially constant resistance to flow of cerebrospinal fluids through the shunt body of from 2 to 7 mm Hg/ml/min. In another, equally preferred embodiment, the flow restricting component of the shunt body is capable of maintaining a passive and essentially constant resistance to flow of cerebrospinal fluids through the shunt body of from 2 to 6 mm Hg/ml/min. In another, equally preferred embodiment, the flow restricting component of the shunt body is capable of maintaining a passive and essentially constant resistance to flow of cerebrospinal fluids through the shunt body of from 2 to 5 mm Hg/ml/min. In another, equally preferred embodiment, the flow restricting component of the shunt body is capable of maintaining a passive and essentially constant resistance to flow of cerebrospinal fluids through the shunt body of from 1 to 4 mm Hg/ml/min. In another, equally preferred embodiment, the flow restricting component of the shunt body is capable of maintaining a passive and essentially constant resistance to flow of cerebrospinal fluids through the shunt body of from 4 to less than 8 mm Hg/ml/min.

In another, equally preferred embodiment, the flow restricting component of the shunt body is capable of maintaining a passive and essentially constant resistance to flow of cerebrospinal fluids through the shunt body of a constant value of 0.1 to 0.5 mm Hg/ml/min, such as from 0.5 to 1.0 mm Hg/ml/min, for example from 1.0 to 1.5 mm Hg/ml/min, such as from 1.5 to 2.0 mm Hg/ml/min, for example from 2.0 to 2.5 mm Hg/ml/min, such as from 2.5 to 3.0 mm Hg/ml/min, for example from 3.0 to 3.5 mm Hg/ml/min, such as from 3.5 to 4.0 mm Hg/ml/min, for example from 4.0 to 4.5 mm Hg/ml/min, such as from 4.5 to 5.0 mm Hg/ml/min, for example from 5.0 to 5.5 mm Hg/ml/min, such as from 5.5 to 6.0 mm Hg/ml/min, for example from 6.0 to 6.5 mm Hg/ml/min, such as from 6.5 to 7.0 mm Hg/ml/min, for example from 7.0 to 7.5 mm Hg/ml/min, such as from 7.5 to less than 8.0 mm Hg/ml/min, for example from 0.1 to 1 mm Hg/ml/min, such as from 1 to 2 mm Hg/ml/min, for example from 2 to 3 mm Hg/ml/min, such as from 3 to 4 mm Hg/ml/min, for example from 4 to 5 mm Hg/ml/min, such as from 5 to 6 mm Hg/ml/min, for example from 6 to 7 mm Hg/ml/min, such as from 7 to less than 8 mm Hg/ml/min, for example from 0.1 to 2 mm Hg/ml/min, such as from 2 to 4 mm Hg/ml/min, for example from 4 to 6 mm Hg/ml/min, such as from 6 to less than 8 mm Hg/ml/min, for example from 0.1 to 2.5 mm Hg/ml/min, such as from 2.5 to 5.0 mm Hg/ml/min, for example from 5.0 to 7.5 mm Hg/ml/min, such as from 3.0 to 7.0 mm Hg/ml/min, for example from 3.5 to 6.5 mm Hg/ml/min, such as from 4.0 to 6.0 mm Hg/ml/min, for example from 4.5 to 5.5 mm Hg/ml/min, such as about 5.0 mm Hg/ml/min.

Preferably, the flow restricting component of the shunt body is selected from the group consisting of a tubular structure, a plurality of tubular structures, a porous mass, a fibrous mass, a structure being restricted by co-extending fibres arranged therein, and a structure being restricted by co-extending rods arranged therein, although any structure capable of maintaining an essentially constant resistance to flow is envisaged as being within the scope of the present invention. In one embodiment, said flow restricting component may be made from one or more material capable of maintaining a passive and essentially constant resistance to flow; more preferably said brain ventricle catheter and/or sinus catheter is comprised of an adhesion-resistant and/or infection-resistant material. More preferably, said material is biocompatible. Example of preferred materials include one or more of: a silicone elastomer, teflon, HD polyethylene, such as gas sterilized polypropylene, polysulfone, polystyrene, PVC, nylon, titanium, shape memory alloys such as Nitinol or polyethersulfone.

The length of the flow restricting compartment is vital for generating the desired level of resistance to flow, and can be calculated according to the law of Hagen-Poiseulle taking into consideration the required resistance to CSF-outflow. In particularly preferred embodiments, the internal radius of the tubular flow passage restricting means is more than 0.05 mm and preferably less than 0.50 mm, for example a tubular structure having an internal radius of about 0.06 mm, for example about 0.07 mm, such as about 0.08 mm, for example about 0.09 mm, such as about 0.10 mm, for example about 0.11 mm, such as about 0.12 mm, for example about 0.13 mm, such as about 0.14 mm, for example about 0.15 mm, such as about 0.16 mm, for example about 0.17 mm, such as about 0.18 mm, for example about 0.19 mm, such as about 0.20 mm, for example about 0.21 mm, such as about 0.22 mm, for example about 0.23 mm, such as 0.24 mm, for example 0.25 mm, such as 0.26 mm, for example 0.27 mm, for example about 0.28 mm, such as about 0.29 mm, for example about 0.30 mm, such as 0.31 mm, for example 0.32 mm, such as 0.33 mm, for example 0.34 mm, for example about 0.35 mm, such as about 0.36 mm, for example about 0.37 mm, such as 0.38 mm, for example 0.39 mm, such as 0.40 mm, for example 0.42 mm, for example about 0.44 mm, such as about 0.46 mm, for example a tubular structure having an internal radius of about 0.48 mm. In another embodiment, the flow restricting component of the shunt body comprises a single tubular structure having an internal diameter of less than 0.2 mm, and appropriate lengths of the flow restricting component can be calculated accordingly, as follows:

L=((ICP-Pss)×7×pi×R⁴)/8×F×V (Hagen-Poiseulle's law), wherein ICP is the intracranial pressure, Pss is the pressure in the sagittal sinus, F is the flow rate of the cerebrospinal fluid and V is the viscosity of the cerebrospinal fluid.

In one preferred embodiment, the length of the flow restricting component is in the range of from about 3.0 mm to about 90 mm, such as from about 3.0 mm to about 80 mm, for example from about 3.0 mm to about 75 mm, such as from about 3.0 mm to about 70 mm, for example from about 3.0 mm to about 65 mm, such as from about 3.0 mm to about 60 mm, for example from about 3.0 mm to about 55 mm, such as from about 3.0 mm to about 50 mm, for example from about 3.0 mm to about 45 mm, such as from about 3.0 mm to about 40 mm, for example from about 3.0 mm to about 35 mm, such as from about 3.0 mm to about 30 mm, for example from about 3.0 mm to about 25 mm, such as from about 3.0 mm to about 22 mm, for example from about 3.0 mm to about 20 mm, such as from about 3.0 mm to about 18 mm, for example from about 3.0 mm to about 16 mm, such as from about 3.0 mm to about 14 mm, for example from about 3.0 mm to about 12 mm, such as from about 3.0 mm to about 10 mm, for example from about 10 mm to about 90 mm, such as from about 10 mm to about 80 mm, for example from about 10 mm to about 75 mm, such as from about 10 mm to about 70 mm, for example from about 10 mm to about 65 mm, such as from about 10 mm to about 60 mm, for example from about 10 mm to about 55 mm, such as from about 10 mm to about 50 mm, for example from about 10 mm to about 45 mm, such as from about 10 mm to about 40 mm, for example from about 10 mm to about 35 mm, such as from about 10 mm to about 30 mm, for example from about 10 mm to about 25 mm, such as from about 10 mm to about 20 mm, for example from about 10 mm to about 15 mm, such as about 10 mm, for example about 15 mm, such as about 20 mm, for example about 22 mm, such as about 24 mm, for example about 26 mm, such as about 20 mm, for example about 22 mm, such as about 24 mm, for example about 26 mm, such as about 28 mm, for example about 30 mm, such as about 32 mm, for example about 34 mm, such as about 36 mm, for example about 38 mm, such as about 40 mm, for example about 45 mm, such as about 50 mm, for example about 55 mm, such as about 60 mm, for example about 65 mm, such as about 70 mm, for example about 75 mm, such as about 80 mm, for example about 85 mm.

In another embodiment of the present invention, the total length of the at least one tubular structure of the flow restricting component is divided into two or more individual segments.

Shunt Location

In one embodiment of the present invention, cerebrospinal fluid is shunted from a brain ventricle to either or both of the two large venous sinuses of the cranium that begin at the bony protuberance on the middle of the inner surface of the occipital bone at the intersection of its bony ridges and terminate at the jugular foramen on either side. More preferably, the cerebrospinal fluid is shunted from a brain ventricle to the sagittal sinus. In an equally preferred embodiment of the present invention, the cerebrospinal fluid is shunted from the brain ventricle and to the transverse sinus.

Shunt Body

In one preferred embodiment of the present invention, the shunt body of the shunt system comprises at least one check valve for preventing cerebrospinal fluid present in the sinus catheter or cerebrospinal fluid, having been shunted to the sinus system of the individual, from flowing back from the sinus catheter or from the sinus system to the shunt body or to the brain ventricle catheter. Preferably, said at least one check valve does not have any inherent resistance or opening pressure, and essentially does not exert any resistance on the flow of cerebrospinal fluid from the brain ventricle catheter through the shunt body to the sinus catheter. More preferably, the resistance to flow through the shunt body is independent of said at least one check valve and defined solely by the flow resistance of the flow restricting component. In the most preferred embodiment, the operation of said at least one check valve is independent of a predetermined opening pressure to be overcome by the differential pressure defined by the difference between the intracranial pressure and the pressure in the sinus. Preferably, said at least one check valve comprises a ball valve and optionally further comprises valve members selected from the group consisting of guided rigid valve members and flexible valve members, including rigid, ring shaped valve members, and flexible valve members such as tongue-shaped laminae. In one preferred embodiment of the present invention, said at least one check valve comprises a mitral silicone valve. Preferably, said sat least one check valve comprises components made out of one or more of rubber, Stellite alloy, titanium, stainless steel, turbostratic carbons such as pyrolytic carbon, or silicone rubber components, optionally coated with a biocompatible coating such as titanium nitride or turbostratic carbons such as pyrolytic carbon.

### PREFERRED EMBODIMENTS OF THE SHUNT SYSTEM

The shunt system preferably comprises a shunt body (10), preferably made from silicone rubber, an antechamber (11) having opposite flat walls (12), preferably made from hard silicone rubber, and opposite domed walls (13), preferably made from soft, perforatable, self-healing silicone rubber. Preferably, at the proximal end (the top end) of the shunt body, the chamber walls end in a tapering end comprising a tip (14), to which a brain ventricle catheter (15) can be connected and secured.

Preferably, the antechamber (11) is connected to the tubular flow restricting component (16) so that the distal end of the chamber (11) forms an inlet to a tubular flow restricting component (16). Preferably, a check valve or non-return valve (17) is arranged both at the entrance to the antechamber (11) and at the outlet of the tubular flow restricting component (16).

In one preferred embodiment of the present invention, fluidic connection to the sinus of the individual is provided by a tubular drain (18), and fluidic connection to a brain ventricle of the individual is provided by a brain ventricle catheter (15). The brain ventricle catheter (15) is preferably attached to the tip or inlet connector (14), which is provided with an annular bead, and the brain ventricle catheter is optionally secured by means of a ligature. Preferably, the length of the connector (14) is about 5 mm. In one preferred embodiment of the present invention, the tubular flow restricting component (16) is dimensioned in accordance with Hagen-Poiseulle's law so as to provide a passive and substantially constant resistance to flow of less than 8 mm Hg/ml/min. Preferably, the tubular flow restricting component is substantially straight. Preferably, the inner walls of the flow restricting component are substantially smooth. The material from which the walls of the tubular flow restricting component are made is preferably selected from the group consisting of titanium, hard silicone rubber, HD polyethylene, such as gas sterilized polypropylene, polycarbonate, polysulfone, polystyrene, PVC and titanium, vanadium steels, aluminium, stainless steels, teflon, silastic, polyethylene, titanium alloys, and ultra-high molecular weight polyethylene/metal combinations. The tubular drain (18) for the sinus is preferably made from titanium or silicone rubber.

In one preferred embodiment of the invention, the distal 5 mm of the tubular drain (18) has an outer diameter of 2 mm and an inner diameter of 1.5 mm, and the part of the drain that goes through the skull has generally an outer diameter of 3 mm and an inner diameter of 1.5 mm. Furthermore, it is preferred that the distance of the part of the drain with the largest diameter can be regulated so as to fit the distance from the shunt body to the hole over the sagittal sinus. Preferably the tubular drain (18) comprises a first tube, preferably comprised of titanium tube, an inner diameter of 1.5 mm and a length of about 20 mm, attached to a second tube, preferably comprised of silicone rubber, with and outer/inner diameter of 3/1.5 mm, and a length of about 60 mm.

Preferably, the tubular drain (18) further comprises a stilet for guiding the silicone rubber tube through a borehole in the skull of the individual.

In a preferred embodiment of the present invention, the shunt system is capable of shunting CSF at a constant flow rate. Preferably, said constant flow rate is in the range of from 40 ml per hour to 140 ml per hour. In another preferred embodiment of the present invention, the constant flow rate is about 40 ml per hour, such as about 45 ml/hour, for example 50 ml per hour, such as about 55 ml/hour, for example about 60 ml per hour, such as about 65 ml/hour, for example about 70 ml per hour, such as about 75 ml/hour, for example about 80 ml per hour, such as about 85 ml/hour, for example about 90 ml per hour, such as about 95 ml/hour, for example 100 ml per hour, such as about 105 ml/hour, for example about 110 ml per hour, such as about 115 ml/hour, for example about 120 ml per hour, such as about 125 ml/hour, for example about 130 ml per hour, such as about 135 ml/hour, for example about 140 ml per hour, such as from 40 to 50 ml per hour, for example from 50 to 60 ml per hour, such as from 60 to 70 ml per hour, for example from 70 to 80 ml per hour, such as from 80 to 90 ml per hour, for example from 90 to 100 ml per hour, such as from 110 to 120 ml per hour, for example from 120 to 130 ml per hour, such as from 130 to 140 ml per hour.

Preferably, the intercranial pressure of the individual is in the range of from −170 mm Hg to 200 mm Hg.

There is also provided the use of a shunt body comprising a flow restricting component capable of maintaining a passive and essentially constant resistance to outflow of CSF through the shunt body, in the manufacture of a shunt system according to the present invention, such as for the treatment of an individual suffering from, or at risk of suffering from, a condition related to the retention and/or accumulation of toxic substances in brain tissue and/or the CSF space. Preferably, said condition is Alzheimer's disease.

The coated shunt system according to the invention can preferably be used for shunting toxic substances present in brain tissue and/or the CSF space to the sinus system of an individual suffering from, or at risk of developing, a condition related to the retention and/or accumulation of toxic substances in brain tissue and/or the CSF space.

Method for Implanting Different Catheters of a Cerebrospinal Fluid Shunt System

In another preferred embodiment of the present invention, a method is provided for implanting different catheters of a cerebrospinal fluid shunt system into a brain ventricle and the sinus system, respectively, of an individual. The first step in said method comprises providing a shunt system as described herein. The shunt body of said shunt system is placed subcutaneously on top of the calvarium of an individual, preferably behind the coronal suture on one side of the sagittal suture. The second end of the brain ventricle catheter is inserted in a brain ventricle via a first borehole, and a first end of the brain ventricle catheter is connected to a first location on the shunt body. A second end of the sinus catheter is inserted into the sinus system of the individual via a second borehole, and a first end of the sinus catheter is connected to a second location on the shunt body. Said shunt body provides fluidic communication between the brain ventricle catheter and the sinus catheter. Preferably, said second end of the sinus catheter is inserted via the second borehole into one of the two large venous sinuses of the cranium that begin at the bony protuberance on the middle of the inner surface of the occipital bone at the intersection of its bony ridges and terminate at the jugular foramen on either side. Said large venous sinus is preferably the sagittal sinus via the second borehole. Equally preferably, said large venous sinus is the transverse sinus. It is preferred that the second end of the brain ventricle catheter is inserted into the right brain ventricle via the first borehole. Equally preferred is insertion of the second end of the brain ventricle catheter into the left brain ventricle via the first borehole. Preferably, said method for implanting different catheters of a cerebrospinal fluid shunt system into a brain ventricle and the sinus system comprises the further step of shunting cerebrospinal fluid from a brain ventricle and to either one or both of the two large venous sinuses of the cranium that begin at the bony protuberance on the middle of the inner surface of the occipital bone at the intersection of its bony ridges and terminate at the jugular foramen on either side. More preferably, the cerebrospinal fluid is shunted from the brain ventricle and to the sagittal sinus. Equally preferably, the cerebrospinal fluid is shunted from the brain ventricle and to the transverse sinus. It is preferred that in the this further step of shunting cerebrospinal fluid from a brain ventricle, the resistance to flow through the flow restricting component of the shunt body is from 2 to less than 8 mm Hg/ml/min. More preferably, said resistance to flow through the flow restricting component of the shunt body is from 4 to 6 mm Hg/ml/min. More preferably, said resistance to flow through the flow restricting component of the shunt body is about 5 mm Hg/ml/min.

Method for Shunting Cerebrospinal Fluid from a Brain Ventricle to the Sinus System of an Individual

In another embodiment of the present invention, a method is provided for shunting cerebrospinal fluid from a brain ventricle to the sinus system of an individual. The first step in said method is to provide a shunt system as disclosed herein, and inserting the first catheter into a brain ventricle of the individual to drain cerebrospinal fluid from the brain ventricle. The second catheter is inserted into the sinus system of the individual to feed the cerebrospinal fluid via the shunt body into the sinus system, and the brain ventricle catheter is connected to a first location on the shunt body. The sinus catheter is connected to a second location on the shunt body, whereby the shunt member provides fluidic communication between the first and second catheters. Cerebrospinal fluid is shunted from a brain ventricle to the sinus system of an individual, whereby the shunt member provides fluidic communication between the first and second catheters. It is preferred that cerebrospinal fluid is shunted from a brain ventricle and to either one or both of the two large venous sinuses of the cranium that begin at the bony protuberance on the middle of the inner surface of the occipital bone at the intersection of its bony ridges and terminate at the jugular foramen on either side. More preferably, said large venous sinus is the sagittal sinus. Equally preferably, said large venous sinus is the transverse sinus. In one preferred embodiment of the method of shunting cerebrospinal fluid of the present invention, the resistance to flow through the flow restricting component of the shunt body is from 2 to less than 8 mm Hg/ml/min. More preferably, said resistance is from 4 to 6 mm Hg/ml/min. More preferably, said resistance is about 5 mm Hg/ml/min. Said method for shunting cerebrospinal fluid, preferably comprises the further step of preventing cerebrospinal fluid from flowing back from the second catheter to the first catheter by introducing at least one check valve into the shunt body. In another preferred embodiment of said method for shunting cerebrospinal fluid, the cerebrospinal fluid is shunted through at least one flow passage structure having an internal radius of about 0.20 mm. In another preferred embodiment of said method for shunting cerebrospinal fluid, the flow rate of shunted cerebrospinal fluid is constant. Said constant flow rate is preferably in the range of from 40 ml per hour to 140 ml per hour. More preferably, said constant flow rate is at least 40 ml per hour, such as about 45 ml/hour, for example 50 ml per hour, such as about 55 ml/hour, for example about 60 ml per hour, such as about 65 ml/hour, for example about 70 ml per hour, such as about 75 ml/hour, for example about 80 ml per hour, such as about 85 ml/hour, for example about 90 ml per hour, such as about 95 ml/hour, for example 100 ml per hour, such as about 105 ml/hour, for example about 110 ml per hour, such as about 115 ml/hour, for example about 120 ml per hour, such as about 125 ml/hour, for example about 130 ml per hour, such as about 135 ml/hour, for example about 140 ml per hour, such as from 40 to 50 ml per hour, for example from 50 to 60 ml per hour, such as from 60 to 70 ml per hour, for example from 70 to 80 ml per hour, such as from 80 to 90 ml per hour, for example from 90 to 100 ml per hour, such as from 110 to 120 ml per hour, for example from 120 to 130 ml per hour, such as from 130 to 140 ml per hour. In one preferred embodiment of the methods for shunting cerebrospinal fluid, the intercranial pressure of the individual is in the range of from −170 mm Hg to 200 mm Hg.

The methods disclosed herein are also envisaged as being used in combination with other medical treatments, for instance conventional drug treatments. By “in combination”, it is meant that the methods disclosed herein may be used on an individual prior to, during, or after treatment of the individual with one or more other medical treatment.

Said medical treatment may comprise administration of a compound inside the lumen of said shunt. In one preferred embodiment, an individual is treated with the methods disclosed herein, in combination with administration of one or more of an antibiotic, anti-coagulants such as heparin, Acetazolamide or Frusemide, Isosorbide, Glycerol, Urokinase, Vancomycine, calcification inhibiting agents or MEDTA. in another, equally preferred embodiment, an individual is treated with the methods disclosed herein, in combination with administration of one or more of an anti-infective compound such as vancomycin, EDTA, Gentamycin, Chymotrypsin, chlorine dioxide, or Minocycline. It is also envisaged that the shunt system of the present invention may be adapted to be capable of being infused with a drug, thus allowing ease of drug delivery. The shunt system may also be impregnated with bioactive compounds, such as a drug, before being positioned inside the individual.

1. A system for shunting cerebrospinal fluids from a brain ventricle to the sinus system of an individual, said system comprising:
i) a shunt body allowing fluid communication between a brain ventricle and a part of the sinus system of the individual,
wherein said shunt body comprises a flow restricting component capable of maintaining a passive and essentially constant resistance to flow of cerebrospinal fluids through the shunt body,
ii) a brain ventricle catheter connected to the shunt body at a first location thereof,
wherein the brain ventricle catheter is capable of draining cerebrospinal fluids from a brain ventricle to the shunt body, and
iii) a sinus catheter connected to the shunt body at a second location thereof,
wherein the sinus catheter is capable of draining, to the sinus system of the individual, cerebrospinal fluids having been drained from the brain ventricle and passed through the flow restricting component of the shunt body to the sinus catheter,
wherein i) the internal or external surface of the shunt body, or ii) the internal or external surface of the brain ventricle catheter, or iii) the internal or external surface of the sinus catheter, comprises a biocompatible and/or hemocompatible material comprising an inert surface preventing biological material from maintaining longer lasting contact with the inert surface, wherein said hemocompatible material is optionally coated with a plurality of charged species capable of increasing the hemocompatibility of the surface.

2. The shunt system for shunting cerebrospinal fluids according to claim 1, wherein the flow restricting component is capable of maintaining a resistance to flow of cerebrospinal fluids of a constant value of from 0.1 to less than 8 mm Hg/ml/min.

3-4. (canceled)

5. The shunt system for shunting cerebrospinal fluids according to claim 1, wherein the flow restricting component is capable of maintaining a passive resistance to flow of cerebrospinal fluids of a constant value of from 2 to less than 8 mm Hg/ml/min.

6-19. (canceled)

20. The shunt system for shunting cerebrospinal fluids according to claim 1, wherein the flow restricting component is capable of maintaining a passive resistance to flow of cerebrospinal fluids of a constant value of from 2 to 7 mm Hg/ml/min.

21-23. (canceled)

24. The shunt system for shunting cerebrospinal fluids according to claim 1, wherein the flow restricting component is capable of maintaining a passive resistance to flow of cerebrospinal fluids of a constant value of from 4 to less than 8 mm Hg/ml/min.

25. (canceled)

26. The shunt system according to claim 1 wherein the flow restricting component is selected from the group consisting of a tubular structure, a plurality of tubular structures, a porous mass, a fibrous mass, a structure being restricted by co-extending fibres arranged therein, and a structure being restricted by co-extending rods arranged therein.

27. The shunt system according to claim 1 wherein the flow restricting component comprises at least one tubular structure having an internal radius of more than 0.05 mm and less than 0.50 mm.

28. The shunt system according to claim 26, wherein the flow restricting component comprises a single tubular structure having an internal diameter of less than 0.2 mm.

29. The shunt system according to claim 26, wherein the length of the at least one tubular structure is in the range of from about 3.0 mm to about 90 mm.

30. The shunt system according to claim 29, wherein the total length of the at least one tubular structure is divided in two or more individual segments.

31. The shunt system according to claim 1 further comprising at least one check valve located within the shunt body for preventing cerebrospinal fluid from flowing back from the sinus catheter to the brain ventricle catheter.

32. The shunt system according to claim 31, wherein said at least one check valve does not have any inherent resistance or opening pressure and essentially does not exert any resistance on the flow of cerebrospinal fluid through the shunt body.

33. The shunt system according to claim 31, wherein the resistance to flow through the shunt body is independent of said at least one check valve and defined solely by the flow resistance of the flow restricting component.

34. The shunt system according to claim 31, wherein the operation of said at least one check valve is independent of a predetermined opening pressure to be overcome by the differential pressure defined by the difference between the intracranial pressure and the pressure in the sinus.

35. The shunt system according to claim 31, wherein said at least one check valve comprises a ball valve and optionally further comprises valve members selected from the group consisting of guided rigid valve members and flexible valve members, including rigid, ring shaped valve members, and flexible valve members optionally as tongue-shaped laminae.

36. The shunt system according to claim 31, wherein said at least one check valve comprises a mitral silicone valve.

37. The shunt system according to claim 1, wherein the brain ventricle catheter is connected to a first end location of said shunt body, and wherein said sinus catheter is connected to a second end location of said shunt body.

38. The shunt system according to claim 1 further comprising a shunt body (10) made from silicone rubber, an antechamber (11) having opposite flat walls (12) made from hard silicone rubber, and opposite domed walls (13) made from soft, perforatable, self-healing silicone rubber,
wherein at the proximal end (the top end) the chamber walls end in a tapering end comprising a tip (14), to which a brain ventricle catheter (15) can be connected and secured,
wherein the antechamber (11) is connected to the tubular flow restricting component (16) so that the distal end of the chamber (11) forms an inlet to a tubular flow restricting component (16),
wherein a check valve or non-return valve (17) is arranged both at the entrance to the antechamber (11) and at the outlet of the tubular flow restricting component (16),
wherein fluidic connection to the sinus system of the individual is provided by a tubular drain (18), and
wherein fluidic connection to a brain ventricle of the individual is provided by a brain ventricle catheter (15).

39-47. (canceled)

48. A method for implanting different catheters of a cerebrospinal fluid shunt system into a brain ventricle and the sinus system, respectively, of an individual, said method comprising the steps of
i) providing a shunt system according to claim 1,
ii) placing the shunt body of the shunt system subcutaneously on top of the calvarium of an individual, optionally behind the coronal suture on one side of the sagittal suture,
iii) inserting a second end of the brain ventricle catheter in a brain ventricle via a first borehole,
iv) optionally connecting a first end of the brain ventricle catheter to a first location on the shunt body;
v) inserting a second end of the sinus catheter into the sinus system of the individual via a second borehole,
vi) optionally connecting a first end of the sinus catheter to a second location on the shunt body,
wherein the shunt body provides fluidic communication between the brain ventricle catheter and the sinus catheter.

49-59. (canceled)

60. A method for shunting cerebrospinal fluid from a brain ventricle to the sinus system of an individual, said method comprising the steps of
i) providing a shunt system according to claim 1,
ii) inserting the first catheter into a brain ventricle of the individual to drain cerebrospinal fluid from the brain ventricle,
iii) inserting the second catheter into the sinus system of the individual to feed the cerebrospinal fluid via the shunt body into the sinus system,
iv) shunting cerebrospinal fluid from a brain ventricle to the sinus system of an individual
wherein the shunt member providing fluidic communication between the first and second catheters,

61-84. (canceled)
